(19) United States
(12) Patent Application Publication
(10) Pub. No.: US 2013/0053566 A1
(43) Pub. Date: Feb. 28, 2013

(54) CATIONIC PALLADIUM COMPLEXES COMPRISING DIAMINO CARBENE LIGANDS AND THEIR USE IN CATALYSIS

(71) Applicants: Kamaluddin Abdur-Rashid, Mississauga (CA); Dino Amoroso, Binbrook (CA); Chi-Wing Tsang, Toronto (CA); Wenli Jia, Mississauga (CA)

(72) Inventors: Kamaluddin Abdur-Rashid, Mississauga (CA); Dino Amoroso, Binbrook (CA); Chi-Wing Tsang, Toronto (CA); Wenli Jia, Mississauga (CA)

(21) Appl. No.: 13/641,168

(22) PCT Filed: Apr. 14, 2011
(86) PCT No.: PCT/CA2011/000414

§ 371 Date: Oct. 15, 2012

Related U.S. Application Data
(60) Provisional application No. 61/324,022, filed on Apr. 14, 2010.

(51) Int. Cl. C07F 15/00; C07C 41/30; C07C 2/86; C07C 67/30

(52) U.S. Cl. 546/12; 548/103; 556/137; 560/64; 568/628; 585/454

(57) ABSTRACT

Cationic palladium catalysts comprising diamino carbene ligands, wherein the catalysts are of the formula [Pd(X)_{q}(LBX)ₜ(DC)]^{r+}[Y^{m-}]ₚ or [Pd(X)_{q}(LB)ₙ(LBX)ₜ(DC)]₂^{a+}[V⁻]u[Z²⁻]y, wherein DC is a diamino carbene ligand, X is an anionic ligand, LBX is a combined anionic and neutral ligand, and Y, V, and Z are non-coordinating anions. The compounds are useful in catalytic reactions, including cross-coupling reactions and hydroamination reactions. In particular, the catalysts are used in the following reactions: Suzuki-Miyaura coupling, Kumada coupling, Negishi coupling, Sonogashira coupling, Hartwig-Buchwald amination, and Heck-Mizoroki coupling.

### FIELD OF THE DISCLOSURE

The present disclosure relates to cationic palladium precatalysts comprising diamino carbene ligands.

### BACKGROUND OF THE DISCLOSURE

The utility of palladium-catalyzed coupling processes was quickly recognized by the synthetic community to be highly useful and as such significant effort has been invested to broaden the scope and improve the utility of such processes. The various coupling methodologies that have been established find highly valued applications in the synthesis of natural products and pharmaceuticals,¹ as well as compounds relevant to materials chemistry.²

The typical catalytic cycle for a Pd-catalyzed coupling proceeds through oxidative addition of the electrophilic compounds to the Pd(0) active species followed by transmetallation which is in turn followed by reductive elimination from the Pd(II) intermediate to give the coupled product and the original Pd(0) species which re-enters the cycle. Typically, Pd(II) pre-catalysts are employed as more stable and convenient sources of the normally air-sensitive Pd(0) active species.

### SUMMARY OF THE DISCLOSURE

While Pd(II) pre-catalysts are known in the art, cationic diamino carbene Pd(II) pre-catalysts have not been investigated. Accordingly, the present disclosure includes a cationic palladium pre-catalyst compound of the formula I:

[Pd(X)_{q}(LB)ₙ(LBX)ₜ(DC)]^{r+}[Y^{m-}]ₚ (I)

wherein
DC is a diamino carbene ligand,
X is any anionic ligand,
LB is any neutral Lewis base,
LBX is a combined anionic and neutral ligand,
Y is any non-coordinating anion,
q is 0 or 1,
n is 0 to 3,
t is 0 or 1,
r is 1 or 2,
m is 1 or 2,
p is 1 or 2,
wherein the sum of q+r is 2 or t+r is 2,
when t is 1, q is 0,
when r is 1, m and p are both 1, and
when r is 2, either (i) m is 2 and p is 1, or (ii) m is 1 and p is 2,
wherein when p is 2, Y is the same or different.

In another embodiment, the present disclosure also includes dimeric forms of the pre-catalyst compounds having the formula (Ia)

[Pd(X)_{q}(LB)ₙ(LBX)ₜ(DC)]₂^{a+}[V⁻]ᵤ[Z²⁻]_{y} (Ia)

wherein
DC is a diamino carbene ligand,
X is any anionic ligand,
LB is any neutral Lewis base,
LBX is a combined anionic and neutral ligand,
V is any non-coordinating mono-anion,
Z is any non-coordinating di-anion
q is 0 or 1,
n is 0 to 3,
t is 0 or 1,
a is 2 or 4,
u is 0, 2 or 4,
y is 0, 1 or 2,
wherein the sum of q+a is 3 or 4, or t+a is 3 or 4,
when t is 1, q is 0,
when a is 2, either (i) u is 2 and y is 0; or (ii) u is 0 and y is 1; or
when a is 4, either (i) u is 4 and y is 0; (ii) u is 2 and y is 1; or (iii) u is 0 and y is 2;
wherein when u is 2 or 4, V is the same or different, and
when y is 2, Z is the same or different.

In one embodiment, the compounds of the formulae (I) and (Ia) are chiral or achiral.

In another embodiment of the disclosure, the diamino carbene ligand is a compound of the formula (II):

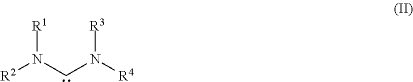

wherein
R¹, R², R³ and R⁴ are independently selected from H, C₁₋₂₀alkyl, C₂₋₂₀alkenyl, C₂₋₂₀alkynyl, C₃₋₂₀cycloalkyl, heteroaryl and aryl, each group being optionally substituted, or
R¹ and R² and/or R³ and R⁴ are linked to form, together with the nitrogen atom to which they are attached, an optionally substituted monocyclic or polycyclic, saturated or unsaturated ring system that contains 3 to 20 carbon atoms, of which one or more of the carbon atoms is optionally replaced with a heteromoiety selected from O, S, NH and NC₁₋₆alkyl, and/or
R¹ and R² or R³ and R⁴ are linked to form, together with the nitrogen atoms to which they are attached, an optionally substituted monocyclic or polycyclic, saturated or unsaturated ring system that contains 3 to 20 carbon atoms, of which one or more of the carbon atoms is optionally replaced with a heteromoiety selected from O, S, NH and NC₁₋₆alkyl,
the optional substituents on R¹, R², R³ and R⁴ are independently selected from one or more of C₁₋₆alkyl, halo, halo-substituted C₁₋₆alkyl, C₃₋₁₀cycloalkyl, aryl and heteroaryl.

In one embodiment, the compounds of the formula (II) are chiral or achiral.

The present disclosure also includes a method of performing metal-catalyzed organic synthesis reactions comprising contacting substrates for the organic synthesis reaction with a cationic palladium precatalyst of the formulae I or Ia as defined above in the presence of a base under conditions for performing the organic synthesis reaction, and optionally isolating one or more products from the organic synthesis reaction. In an embodiment of the disclosure, the organic synthesis reaction is any reaction that benefits from the presence or use of a cationic palladium precatalyst, for example, but not limited to cross-couplings. In an embodiment of the disclosure, the organic synthesis transformation is an asymmetric or chiral synthesis reaction (i.e. provides one enantiomer in excess of the other).

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

The present disclosure will now be described in greater detail with reference to the attached drawings in which:

FIG. 1 is an X-ray crystal structure of precursor A in an embodiment of the disclosure. The thermal ellipsoids were drawn at the 30% probability level, and the hydrogen atoms were omitted for clarity;

FIG. 2 is an X-ray single-crystal structure of precursor B in an embodiment of the disclosure. The thermal ellipsoids were drawn at the 30% probability level, and the hydrogen atoms were omitted for clarity;

FIG. 3 is an X-ray single-crystal structure of precursor C in an embodiment of the disclosure. The thermal ellipsoids were drawn at the 30% probability level, and the hydrogen atoms were omitted for clarity;

FIG. 4 is an X-ray single-crystal structure of IA in an embodiment of the disclosure. The thermal ellipsoids were drawn at the 30% probability level, and the hydrogen atoms were omitted for clarity;

FIG. 5 is an X-ray single-crystal structure of IB.OH₂ in an embodiment of the disclosure. The unit cell contains 1 molecule of IB.OH₂, 1 molecule of BF₄ and 1 molecule of CH₂Cl₂; only IB.OH₂ is shown for clarity. The thermal ellipsoids were drawn at the 30% probability level, and the hydrogen atoms were omitted for clarity;

FIG. 6 is an X-ray single-crystal structure of IC in an embodiment of the disclosure. The unit cell contains 1 molecule of IC, 2 molecules of BF₄ and 1 molecule of CH₂Cl₂; only IC is shown for clarity. The thermal ellipsoids were drawn at the 30% probability level, and the hydrogen atoms were omitted for clarity.

FIG. 7 is an X-ray single-crystal structure of ID in an embodiment of the disclosure. The unit cell contains 1 molecule of ID, 2 molecules of BF₄ and 1 molecule of CH₂Cl₂; only ID is shown for clarity. The thermal ellipsoids were drawn at the 30% probability level, and the hydrogen atoms were omitted for clarity; and

FIG. 8 is an X-ray single-crystal structure of IE in an embodiment of the disclosure. The thermal ellipsoids were drawn at the 30% probability level, and the hydrogen atoms were omitted for clarity.

### DETAILED DESCRIPTION OF THE DISCLOSURE

### (I) Definitions

The term “diamino carbene ligand” as used herein refers to a ligand for palladium which contains a carbon atom having six valence electrons (carbene), in which the carbene carbon atom is bonded to two amino groups. Two of the six valence electrons on the carbene carbon are present as a lone pair, and it is the lone pair which co-ordinates with the palladium atom in the cationic palladium precatalyst in the compounds of the formulae (I) and (Ia). The amino groups may be unsubstituted or substituted with, for example, alkyl groups, alkenyl groups, alkynyl groups, or cycloalkyl groups (all of which are substituted or unsubstituted), or the amino groups may form, together, a heterocyclic ring, or the substituents on the amino groups may form a ring, together with the nitrogen atom.

The term “anionic ligand” as used herein refers to any negatively charged ligand that is commonly used as a ligand in metal catalysis, such as halo (such as chloro), H, C₁₋₆alkoxy and carboxyl (C(═O)O).

The term “neutral Lewis base” as used herein refers to any neutral two electron donor which are optionally present to fulfill the valence requirements of the palladium metal. Examples of neutral Lewis bases include, but are not limited to, acetonitrile and pyridine.

The term “combined anionic and neutral ligand” as used herein refers to any ligand which can act as both an anionic ligand as defined above, as well as a neutral Lewis base, also as defined above. The combined anionic and neutral ligand therefore contains both an anionic moiety (such as an alkoxy, aryloxy or aryl type moiety) and also a neutral moiety which can donate electrons to the palladium to optionally fulfill the valence requirements, such as, but not limited to, an amino group moiety.

The term “non-coordinating anion”, “mono-anion” or “di-anion”, or weakly co-ordinating anion, as used herein refers to any negatively charged ion which acts as a counterion to the positively charged palladium atom. The non-coordinating anion is either a mono-anion or a di-anion, depending on the overall charge of the palladium complex. Examples of non-coordinating mono-anions include, but are not limited to BF₄, B(C₆F₅)₄, or carboranes. Example of non-coordinating di-anions include, but are not limited to CO₃, SO₄ and C₂O₄. It will be understood that depending on the overall charge of the palladium complex, for example a charge of +2, two mono-anions, which are optionally the same or different, balance the positive charge of the palladium, or one alternatively, one di-anion.

The term “chiral” as used herein refers to any of the compounds of the present disclosure, for example compounds of the formulae (I), (Ia) or (II), which contain at least one asymmetric center (chiral atom or chiral center) and thus occur in two non-superimposable mirror-image forms as enantiomers. The term also includes compounds having more than one asymmetric center, such as diastereomers.

The term “C_{1-w}alkyl” as used herein means straight and/or branched chain, saturated alkyl groups containing from one to “w” carbon atoms and includes (depending on the identity of w) methyl, ethyl, propyl, isopropyl, n-butyl, s-butyl, isobutyl, t-butyl, 2,2-dimethylbutyl, n-pentyl, 2-methylpentyl, 3-methylpentyl, 4-methylpentyl, n-hexyl and the like, where the variable w is an integer representing the largest number of carbon atoms in the alkyl group.

The term “C_{2-w}alkenyl” as used herein means straight and/or branched chain, unsaturated alkyl groups containing from two to w carbon atoms and one to three double bonds, and includes (depending on the identity of w) vinyl, allyl, 2-methylprop-1-enyl, but-1-enyl, but-2-enyl, but-3-enyl, 2-methylbut-1-enyl, 2-methylpent-1-enyl, 4-methylpent-1-enyl, 4-methylpent-2-enyl, 2-methylpent-2-enyl, 4-methylpenta-1,3-dienyl, hexen-1-yl and the like, where the variable w is an integer representing the largest number of carbon atoms in the alkenyl group.

The term “C_{2-w}alkynyl” as used herein means straight and/or branched chain, unsaturated alkyl groups containing from two to w carbon atoms and one to three bonds, and includes (depending on the identity of w) propargyl, 2-methylprop-1-ynyl, but-1-ynyl, but-2-ynyl, but-3-ynyl, 2-methylbut-1-ynyl, 2-methylpent-1-ynyl, 4-methylpent-1-ynyl, 4-methylpent-2-ynyl, 2-methylpent-2-ynyl, 4-methylpenta-1,3-diynyl, hexyn-1-yl and the like, where the variable w is an integer representing the largest number of carbon atoms in the alkynyl group.

The term “C_{3-w}cycloalkyl” as used herein means a monocyclic, bicyclic or tricyclic saturated carbocylic group containing from three to w carbon atoms and includes (depending on the identity of w) cyclopropyl, cyclobutyl, cyclopentyl, cyclodecyl and the like, where the variable w is an integer representing the largest number of carbon atoms in the cycloalkyl group.

The term “aryl” as used herein means a monocyclic, bicyclic or tricyclic aromatic ring system containing from 6 to 14 carbon atoms and at least one aromatic ring and includes phenyl, naphthyl, anthracenyl, 1,2-dihydronaphthyl, 1,2,3,4-tetrahydronaphthyl, fluorenyl, indanyl, indenyl and the like.

The term “heteroaryl” as used herein means a monocyclic, bicyclic or tricyclic ring system containing one or two aromatic rings and from 5 to 14 atoms of which, unless otherwise specified, one, two, three, four or five are heteroatoms independently selected from N, NH, N(C₁₋₆alkyl), O and S and includes thienyl, furyl, pyrrolyl, pyrididyl, indolyl, quinolyl, isoquinolyl, tetrahydroquinolyl, benzofuryl, benzothienyl and the like.

The term “halo” as used herein means halogen and includes chloro, fluoro, bromo and iodo.

The term “ring system” as used herein refers to a carbon-containing ring system, that includes monocycles, fused bicyclic and polycyclic rings and bridged rings. Where specified, the carbons in the rings may be substituted or replaced with heteroatoms.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

### (II) Compounds of the Disclosure

The present disclosure relates to cationic Pd(II) pre-catalysts which when converted to the active catalyst, have been determined to be active catalysts in organic reactions, such as coupling reactions or hydroamination reactions, including Suzuki-Miyaura coupling reactions, Negishi coupling (both sp²-sp² and sp²-sp³), Sonogashira coupling, Heck-Mizoroki coupling and Hartwig-Buchwald amination.

Accordingly, the present disclosure includes a cationic palladium pre-catalyst compound of the formula I:

[Pd(X)_{q}(LB)ₙ(LBX)ₜ(DC)]^{r+}[Yⁿ⁻]ₚ] (I)

wherein
DC is a diamino carbene ligand,
X is any anionic ligand,
LB is any neutral Lewis base,
LBX is a combined anionic and neutral ligand,
Y is any non-coordinating anion,
q is 0 or 1,
n is 0 to 3,
t is 0 or 1,
r is 1 or 2,
m is 1 or 2,
p is 1 or 2,
wherein the sum of q+r is 2 or t+r is 2,
when t is 1, q is 0,
when r is 1, m and p are both 1, and
when r is 2, either (i) m is 2 and p is 1, or (ii) m is 1 and p is 2,
wherein when p is 2, Y is the same or different.

In another embodiment, the present disclosure also includes dimeric forms of the pre-catalyst compounds having the formula (Ia)

wherein
DC is a diamino carbene ligand,
X is any anionic ligand,
LB is any neutral Lewis base,
LBX is a combined anionic and neutral ligand,
V is any non-coordinating mono-anion,
Z is any non-coordinating di-anion
q is 0 or 1,
n is 0 to 3,
t is 0 or 1,
a is 2 or 4,
u is 0, 2 or 4,
y is 0, 1 or 2,
wherein the sum of q+a is 3 or 4, or t+a is 3 or 4,
when t is 1, q is 0,
when a is 2, either (i) u is 2 and y is 0; or (ii) u is 0 and y is 1; or
when a is 4, either (i) u is 4 and y is 0; (ii) u is 2 and y is 1; or (iii) u is 0 and y is 2;
wherein when u is 2 or 4, V is the same or different, and
when y is 2, Z is the same or different.

In one embodiment, the precatalyst compounds of the formulae (I) or (Ia) are chiral or achiral, optionally chiral.

In another embodiment of the disclosure, the diamino carbene ligand is a compound of the formula (II):

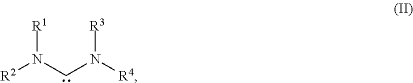

wherein
R³ and R⁴ are independently selected from H, C₁₋₂₀alkyl, C₂₋₂₀alkenyl, C₂₋₂₀alkynyl, C₃₋₂₀cycloalkyl, heteroaryl and aryl, each group being optionally substituted, or
R¹ and R² and/or R³ and R⁴ are linked to form, together with the nitrogen atom to which they are attached, an optionally substituted monocyclic or polycyclic, saturated or unsaturated ring system that contains 3 to 20 carbon atoms, of which one or more of the carbon atoms is optionally replaced with a heteromoiety selected from O, S, NH and NC₁₋₆alkyl, and/or
R¹ and R³ or R² and R⁴ are linked to form, together with the nitrogen atoms to which they are attached, an optionally substituted monocyclic or polycyclic, saturated or unsaturated ring system that contains 3 to 20 carbon atoms, of which one or more of the carbon atoms is optionally replaced with a heteromoiety selected from O, S, NH and NC₁₋₆alkyl,
the optional substituents on R¹, R², R³ and R⁴ are independently selected from one or more of C₁₋₆alkyl, halo, halo-substituted C₁₋₆alkyl, C₃₋₁₀cycloalkyl, aryl and heteroaryl.

In another embodiment, R¹, R², R³ and R⁴ are independently selected from H, C₁₋₁₀alkyl, C₂₋₁₀alkenyl, C₂₋₁₀alkynyl, C₃₋₁₀cycloalkyl, heteroaryl and aryl, each group being optionally substituted. In another embodiment, R¹, R², R³ and R⁴ are independently selected from H, C₁₋₆alkyl, C₂₋₆alkenyl, C₂₋₆alkynyl, C₃₋₆cycloalkyl, heteroaryl and aryl, each group being optionally substituted. In another embodiment, R¹, R², R³ and R⁴ are independently selected from H, C₁₋₄alkyl, C₂₋₄alkenyl, C₂₋₆alkynyl, C₅₋₆cycloalkyl and phenyl, each group being optionally substituted. In another embodiment, R¹, R², R³ and R⁴ are independently selected from H, C₁₋₄alkyl, and phenyl, each group being optionally substituted. In another embodiment, R¹, R², R³ and R⁴ are independently selected from H, methyl, ethyl, propyl, isopropyl, butyl and phenyl, wherein phenyl is substituted at least once, optionally twice, optionally three times by C₁₋₄alkyl. In another embodiment, R¹, R², R³ and R⁴ are isopropyl. In another embodiment, R¹, R², R³ and R⁴ are independently selected from

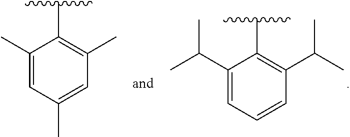

In another embodiment, R¹ and R² or R³ and R⁴ are linked to form, together with the nitrogen atom to which they are attached, an optionally substituted monocyclic or polycyclic, saturated or unsaturated ring system that contains 3 to 10 carbon atoms, of which one or more of the carbon atoms is optionally replaced with a heteromoiety selected from O, S, NH and NC₁₋₆alkyl. In another embodiment, R¹ and R² or R³ and R⁴ are linked to form, together with the nitrogen atom to which they are attached, an optionally substituted monocyclic or polycyclic, saturated or unsaturated ring system that contains 5 to 10 carbon atoms, of which one or more of the carbon atoms is optionally replaced with a heteromoiety selected from O, S, NH and NC₁₋₆alkyl. In another embodiment, R¹ and R² or R³ and R⁴ are linked to form, together with the nitrogen atom to which they are attached, an optionally substituted monocyclic, saturated or unsaturated ring system that contains 5 to 6 carbon atoms, of which one or more of the carbon atoms is optionally replaced with a heteromoiety selected from O, S, NH and NC₁₋₆alkyl.

In another embodiment, R¹ and R³ or R² and R⁴ are linked to form, together with the nitrogen atoms to which they are attached, an optionally substituted monocyclic or polycyclic, saturated or unsaturated ring system that contains 3 to 10 carbon atoms, of which one or more of the carbon atoms is optionally replaced with a heteromoiety selected from O, S, NH and NC₁₋₆alkyl. In another embodiment, R¹ and R³ or R² and R⁴ are linked to form, together with the nitrogen atoms to which they are attached, an optionally substituted monocyclic or polycyclic, saturated or unsaturated ring system that contains 5 to 10 carbon atoms, of which one or more of the carbon atoms is optionally replaced with a heteromoiety selected from O, S, NH and NC₁₋₆alkyl. In another embodiment, R¹ and R³ or R² and R⁴ are linked to form, together with the nitrogen atoms to which they are attached, an optionally substituted monocyclic, saturated or unsaturated ring system that contains 5 to 6 carbon atoms, of which one or more of the carbon atoms is optionally replaced with a heteromoiety selected from O, S, NH and NC₁₋₆alkyl.

In another embodiment of the disclosure, the compound of the formula (II) is

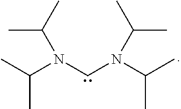

In another embodiment, the compound of the formula (II) is

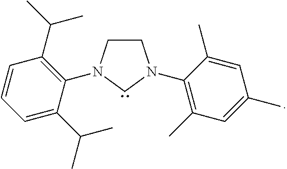

In another embodiment, the optional substituents on R¹, R², R³ and R⁴ are independently selected from one or more, optionally one to five, of C₁₋₄alkyl, halo, halo-substituted C₁₋₄alkyl, C₃₋₆cycloalkyl, aryl and heteroaryl. In another embodiment, the optional substituents on R¹, R², R³ and R⁴ are independently selected from one or more, optionally one to five, of C₁₋₂alkyl, halo, halo-substituted C₁₋₂alkyl, C₆₋₆cycloalkyl and phenyl.

In another embodiment of the disclosure, the compound of the formula (II) is chiral or achiral, optionally chiral.

In an embodiment of the disclosure, X is any suitable anionic ligand, including, for example, halo, H, C₁₋₆alkoxy and carboxyl. In another embodiment, X is Cl.

In another embodiment, LB is any suitable neutral Lewis base, for example any neutral two electron donor, for example acetonitrile or pyridine.

In another embodiment, LBX is any suitable compound that possesses both an anionic moiety and a Lewis base moiety. In another embodiment, LBX is

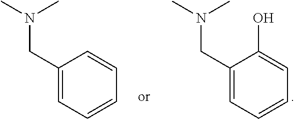

In another embodiment, Y is any non-coordinating counter anion, including, for example, BF₄, B(C₆F₅)₄, a carborane, CO₃, SO₄ and C₂O₄.

In an embodiment of the disclosure, the compound of the formula (I) is

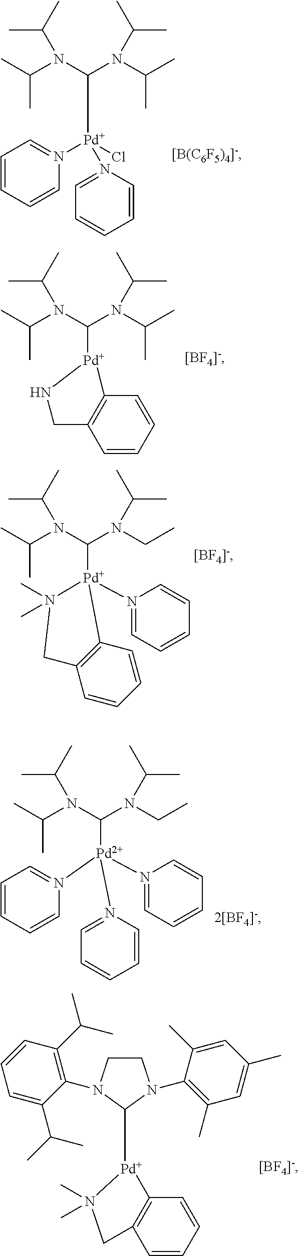

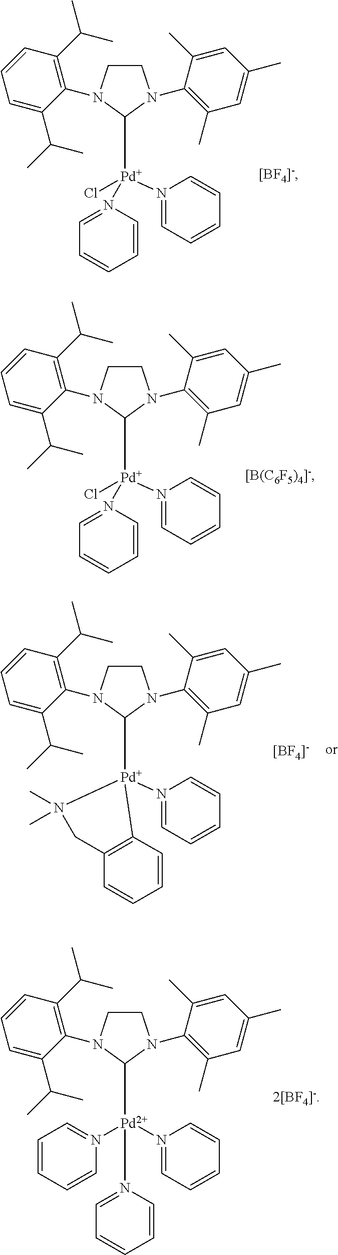

In another embodiment of the disclosure, the compound of the formula (Ia) is

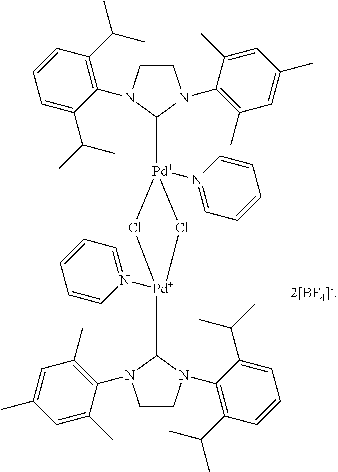

### (III) Preparation of the Compounds of Formulae (I) and (Ia)

In another embodiment of the disclosure, the compounds of the formulae (I) and (Ia) are prepared by the abstraction of an anionic ligand from the corresponding neutral precursors. Accordingly, in an embodiment, the compounds of the formulae (I) and (Ia) are prepared from the corresponding neutral compounds [Pd(X)_{q}(LB)ₙ(LBX)ₜ(DC)] or [Pd(X)_{q}(LB)ₙ(LBX)ₜ(DC)]₂, wherein DC, X, LB, LBX, n and t have the same definitions as described above, and q or t is an integer between 0 and 2.

For example, in an embodiment, neutral precursors of pre-catalyst compounds of the formula (I) are shown below, but not limited to:

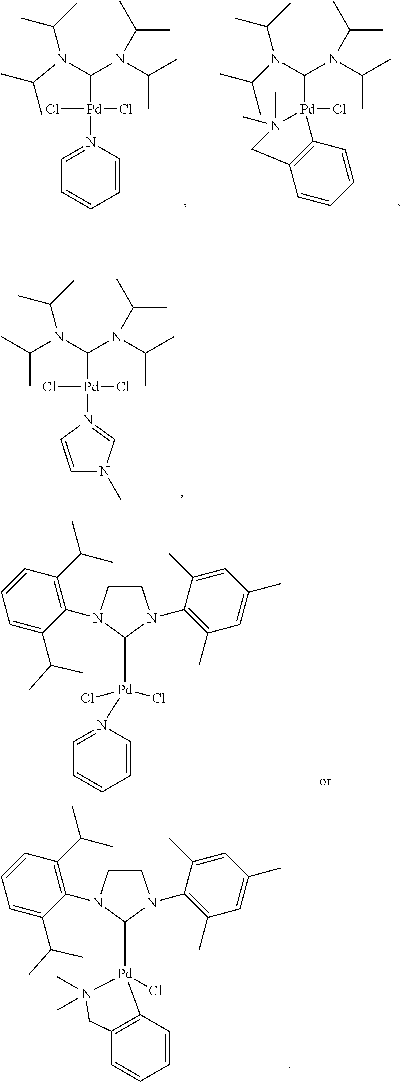

In another embodiment of the disclosure, the neutral precursors of the cationic palladium pre-catalysts compounds of the formula (I) or (Ia) are prepared, for example, as shown in Scheme 1.

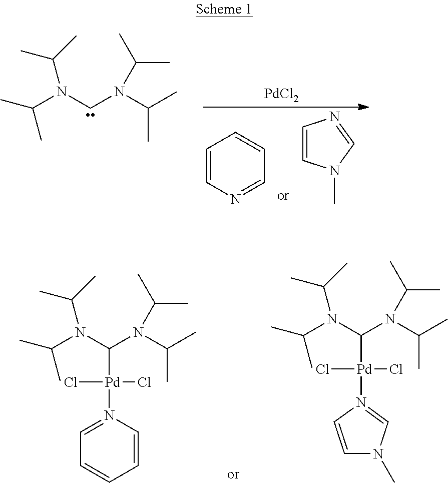

In another embodiment of the disclosure, neutral precursors comprising a combined anionic and neutral ligand are prepared, for example, as shown in Scheme 2.

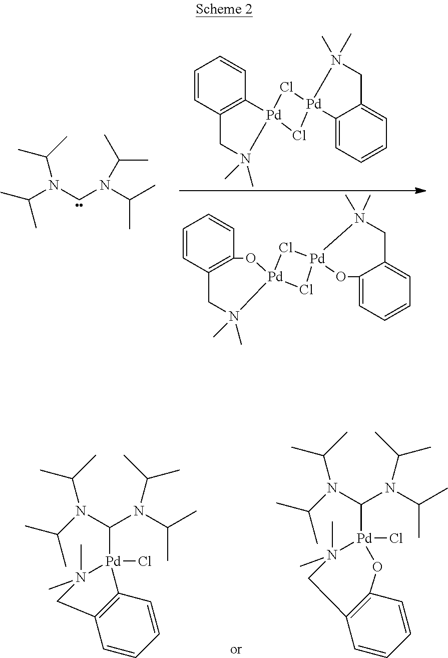

In another embodiment, the compounds of the formula (I) and (Ia) are prepared from the corresponding neutral precursors, for example, by anion abstraction of one or two anionic ligands, with the salt of a weakly or non-coordinating anion, generally as shown in Schemes 3, 4 and 5.

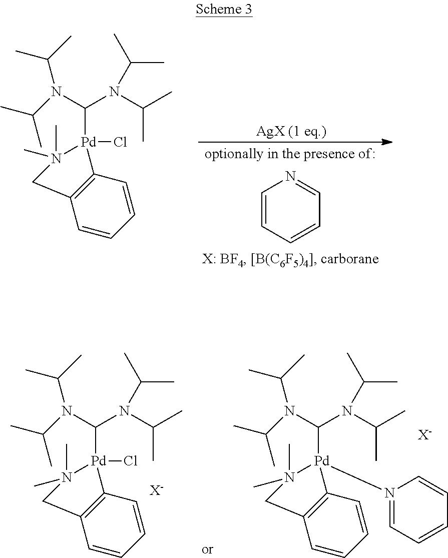

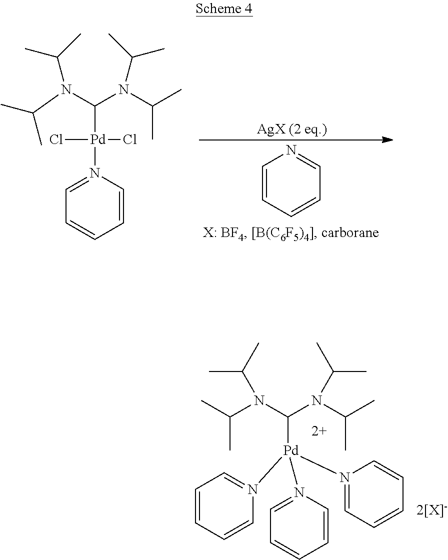

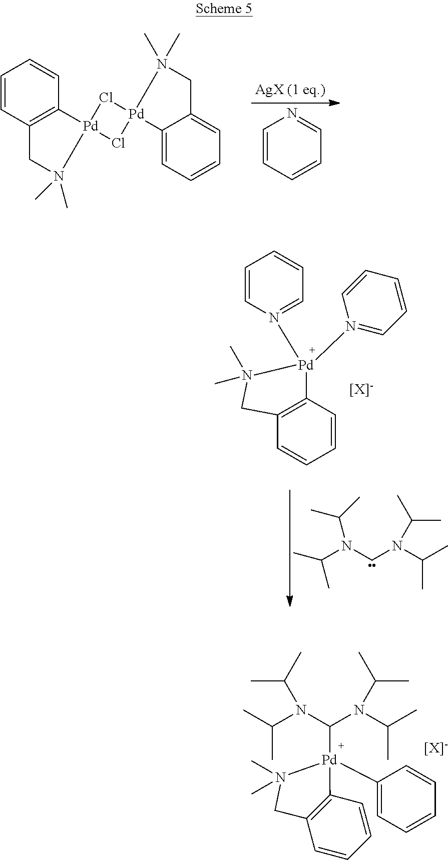

In another embodiment, the compounds of the formula I and (Ia) are prepared generally as shown in Scheme 6.

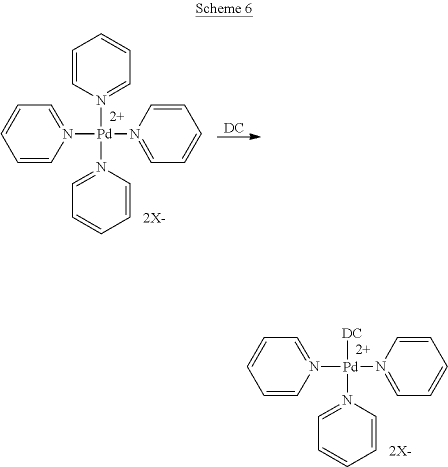

In another embodiment, while specific groups for DC, X, LB and LBX are shown above in Schemes 1-6, a person skilled in the art would appreciate that other equivalent groups as described herein can be substituted to obtain the compounds of the formula (I) and (Ia), in addition to the precursors of these compounds.

### (IV) Methods of the Disclosure

The present disclosure also includes a method of performing palladium-catalyzed organic synthesis reactions comprising contacting substrates for the organic synthesis reaction with cationic palladium precatalyst compounds of the formulae (I) or (Ia) as defined above in the presence of a base under conditions for performing the organic synthesis reaction, and optionally isolating one or more products from the organic synthesis reaction.

In an embodiment of the disclosure, the organic synthesis reaction is any reaction the benefits from the presence or use of a palladium catalyst, for example, but not limited to, cross-couplings and hydroaminations, such as Suzuki-Miyaura coupling reactions, Negishi coupling (both sp²-sp² and sp²-sp³), Sonogashira coupling, and Heck-Mizoroki coupling, as well as Hartwig-Buchwald aminations.

In an embodiment of the disclosure, the organic synthesis transformation is an asymmetric or chiral synthesis reaction (i.e. provides one enantiomer in excess of the other).

In an embodiment of the disclosure, the active palladium catalysts are generated in situ in solution from the compound of the formulae (I) or (Ia) in the presence of a base and the resulting catalyst solution is added to the appropriate starting materials for the organic synthesis transformation.

The following non-limiting examples are illustrative of the present disclosure:

### (V) Examples

### Materials and Methods

Unless indicated otherwise, all chemicals were obtained from Sigma-Aldrich and were used without any other purification unless otherwise specified. All coupling reactions were carried out under inert atmosphere (nitrogen or argon). ¹H and ¹³C NMR spectra were recorded using 200 MHz, 300, 400 and 500 MHz spectrometers. Proton chemical shifts were internally referenced to the residual proton resonance in CDCl₃ (δ 7.26). Carbon chemical shifts were internally referenced to the deuterated solvent signals in CDCl₃ (δ 77.2).

### Example 1

### Synthesis of Precursors

### (i) Precursor A

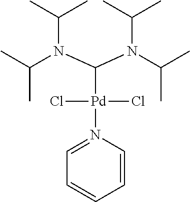

To a 100 mL flask was added PdCl₂ (0.91 g, 5.17 mmol), bis(diisopropylamino)carbene (1.0 g, 4.7 mmol) and pyridine (50 ml) was used to dissolve it. The solution was clear at the beginning and the precipitate slowly formed, together with unreacted PdCl₂. PdCl₂ slowly disappeared during the course of the reaction. The reaction mixture was stirred for 18 hours at room temperature. Then the solvent was completely removed. It was then re-dissolved in CH₂Cl₂ and H₂O was added to wash the product. It was separated, dried over MgSO₄, filtered and concentrated. Et₂O (ca. 200 mL) was then added to form a pale yellow solid. The solid was then filtered off, and the filtrate was concentrated and recrystallized from hexanes to obtain a pale yellow solid as the final product. Yield: 1.4 g, 63%. ¹H NMR (200 MHz, CD₂Cl₂): d8.94 (2H, d, pyridine), 7.76 (1H, t, pyridine), 7.33 (2H, dd, pyridine), 4.83 (4H, m, (CH₃)₂CH₂), 1.63 (24H, d, CH₃).

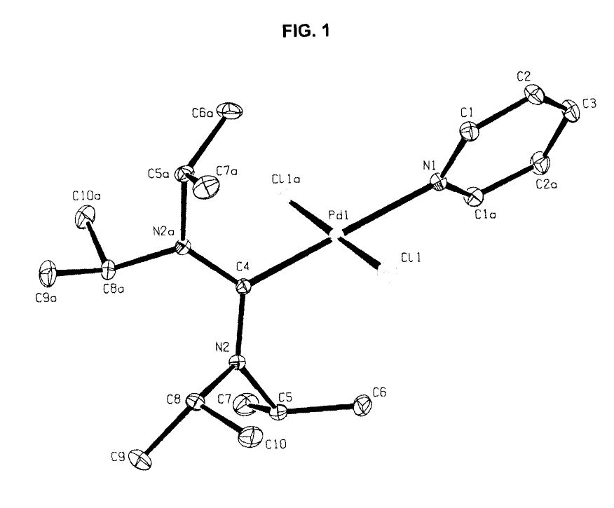

The X-ray crystal structure of Precursor A is shown in FIG. 1.

### (ii) Precursor B

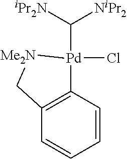

To a 100 mL flask was added Di-μ-chlorobis(N,N)-dimethylbenzylamine)-dipalladium (0.26 g, 0.47 mmol), bis(diisopropylamino)carbene (0.2 g, 0.94 mmol) and the solid mixture was dissolved in THF. The solution was allowed to stir for 18 hours. Then the solvent was concentrated and the product was recrystallized from CH₂Cl₂/hexanes as a pale yellow solid. Yield: 0.4 g, 86%. ¹H NMR (200 MHz, CD₂Cl₂): d 6.95 (2H, d, Ph), 6.90 (1H, m, Ph), 6.80 (1H, m, Ph), 4.74 (4H, m, (CH₃)₂CH₂), 3.77 (2H, s, CH₂), 2.66 (6H, s, CH₃), 1.60 (12H, d, CH₃), 1.49 (12H, d, CH₃).

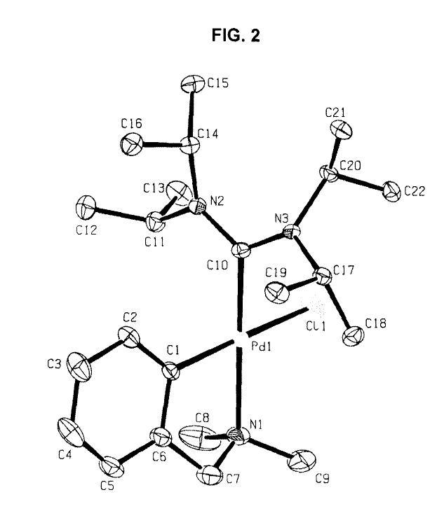

The x-ray crystal structure of Precursor B is shown in FIG. 2.

(iii) Precursor C

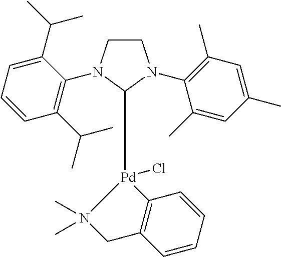

To a flask was added 1-(2,6-diisopropylphenyl)-3-(2,4,6-trimethylphenyl)-4,5-dihydroimidazolium chloride (0.14 g, 0.36 mmol), the Di-μ-chlorobis(N,N)-dimethylbenzylamine)dipalladium (0.10 g, 0.18 mmol) and THF was added to dissolve it. The reaction mixture was allowed to reflux for 18 hours. Then the solution was cooled down and filtered to remove the insoluble solid. Then the solvent was concentrated and the remaining solid was recrystallized from hexanes to obtain a pale yellow solid. Yield: 0.15 g, 66%. ¹H NMR (200 MHz, CD₂Cl₂): δ 6.65-7.35 (9H, m, Ph), 4.05 (4H, m, —NCH₂CH₂N—), 3.58 (2H, m, (CH₃)₂CH₂), 3.40 (2H, s, CH₂), 2.62 (6H, S, CH₃), 2.25 (12H, m, CH₃), 1.50 (3H, d, CH₃), 1.22 (3H, m, CH₃), 0.83 (3H, d, CH₃).

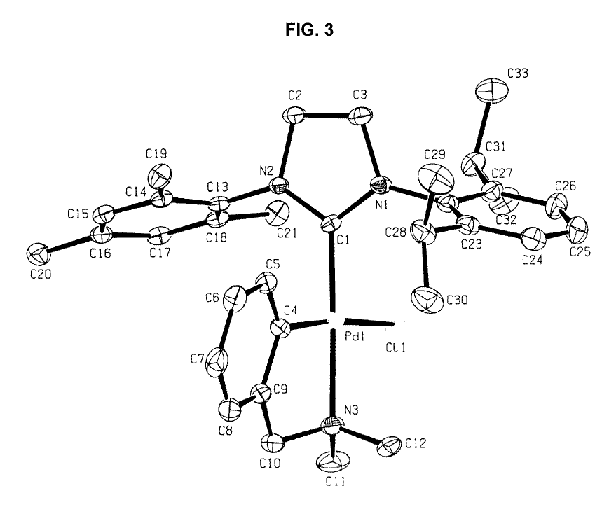

The X-ray crystal structure of Precursor C is shown in FIG. 3.

### (iv) Precursor D

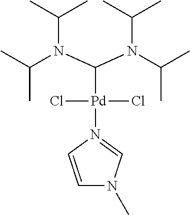

To a 100 mL flask was added PdCl₂ (0.092 g, 0.518 mmol), bis(diisopropyl)carbene (0.1 g, 0.47 mmol), THF (20 ml) and 1-methylimidazole (0.113 ml, 1.4 mmol, 3 equiv.) was added to dissolve it. The solution was clear at the beginning and the precipitate slowly formed, together with unreacted PdCl₂. PdCl₂ slowly disappeared during the course of the reaction. The reaction mixture was stirred for 18 hours at room temperature. Then the solvent was completely removed. It was then re-dissolved in CH₂Cl₂ and H₂O was added to wash the product. It was separated, dried over MgSO₄, filtered and concentrated. Et₂O (ca. 200 mL) was then added to form a pale yellow solid. The solid was then filtered off, and the filtrate was concentrated and recrystallized from hexanes to obtain a pale yellow solid as the final product. Yield: 0.15 g, 70%. ¹H NMR (200 MHz, CD₂Cl₂): δ 7.94 (1H, s, imidazole), 7.36 (1H, s, imidazole), 6.79 (1H, s, imidazole), 4.80 (4H, m, (CH₃)₂CH₂), 3.64 (3H, s, CH₃-imidazole), 1.57 (24H, d, CH₃).

### Example 2

### Preparation of Cationic Palladium Precatalyst Compounds of Formula (I)

### (i) Compound IA

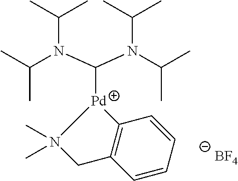

In the glovebox, Precursor B (0.1 g, 0.20 mmol) and AgBF₄ (0.04 g, 0.02 mmol) was mixed together. Then CH₂Cl₂ (4 mL) was added to the solid mixture. The solution immediately turned to a cloudy pale brown solution with the formation of white precipitate. The mixture was allowed to stir for half hour and the solid was filtered off through a syringe filter. The solvent was then concentrated and hexanes was added to the residue. The solid that precipitated was filtered off and the filtrate was concentrated to give the product as a while solid. Yield: 0.049 g, 44%. ¹H NMR (200 MHz, CD₂Cl₂): δ 6.81-6.95 (4H, m, Ph), 4.48 (4H, m, (CH₃)₂CH₂), 3.76 (2H, s, CH₂), 2.68 (6H, s, CH₃), 1.54 (24H, d, CH₃). ¹⁹F NMR (282 MHz, CD₂Cl₂): d −153 (s).

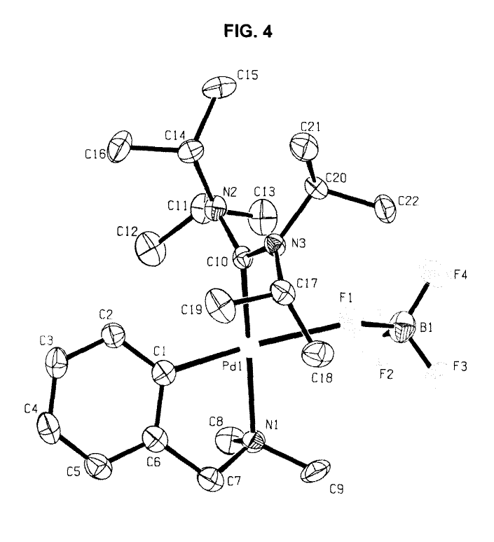

The X-ray crystal structure of compound IA is shown in FIG. 4.

### (ii) Compound IB

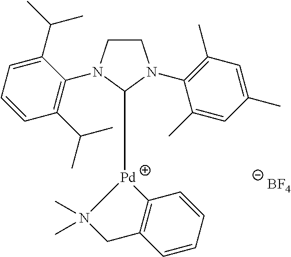

In the glovebox, the Precursor C (0.096 g, 0.15 mmol) and AgBF₄ (0.03 g, 0.15 mmol) were mixed together. Then CH₂Cl₂ (4 mL) was added to the solid mixture. The solution immediately turned to a cloudy yellow solution with the formation of white precipitate. The mixture was allowed to stir for half hour and the solid was filtered off. The filtrate was then concentrated, and the product was recrystallized from hexanes as a pale yellow solid. Yield: 0.086 g, 82%. ¹H NMR (200 MHz, CD₂Cl₂): δ 7.45 (2H, m, Ph), 7.35 (2H, d, Ph), 6.64-6.90 (5H, m, Ph), 4.09 (4H, m, —NCH₂CH₂N—), 3.40 (2H, s, CH₂), 3.25 (2H, m, (CH₃)₂CH₂), 2.38 (6H, s, CH₃), 2.20 (9H, s, CH₃), 1.23 (12H, d, CH₃). ¹⁹F NMR (282 MHz, CD₂Cl₂): δ −153 (s).

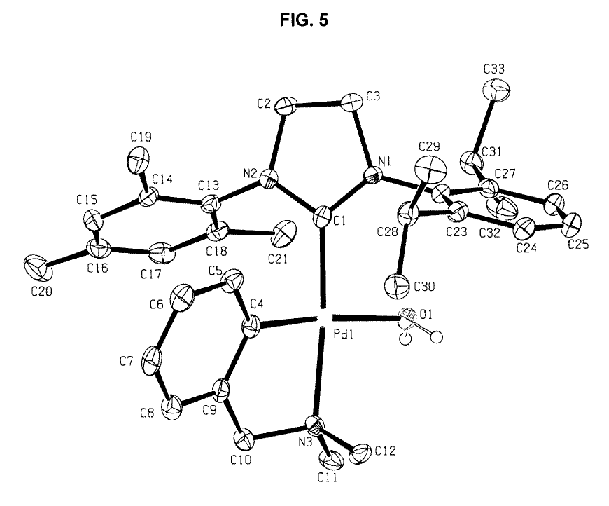

The X-ray crystal structure of compound IB is shown in FIG. 5.

(iii) Compound IC

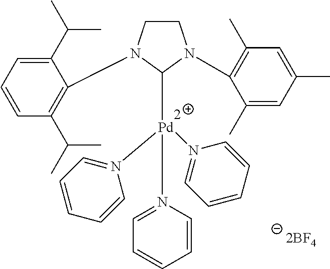

In the glovebox, a small vial was charged with dichloro(pyridine)[trans-(1,3-bis(2,6-diethylphenyl)imidazolin-2-ylidene)]palladium(II) (50 mg, 0.083 mmol) and AgBF₄ (33 mg, 0.165 mmol) and CH₂Cl₂ was added to dissolve the mixture. 4 equiv. of pyridine (0.027 mL) was then added to the solution and the solution was allowed to stir overnight. The solvent was then concentrated to obtain a colorless solid. Yield: 58 mg, 81%. ¹H NMR (300 MHz, CD₂Cl₂): d 6.90-8.60 (20H, m, Ph and pyridine), 4.06 (4H, m, —NCH₂CH₂N—), 3.18 (2H, m, (CH₃)₂CH₂), 2.25 (3H, s, CH₃), 2.12 (6H, s, CH₃), 1.22 (12H, m, (CH₃)₂CH₂). ¹⁹F NMR (282 MHz, CD₂Cl₂): δ −153 (s).

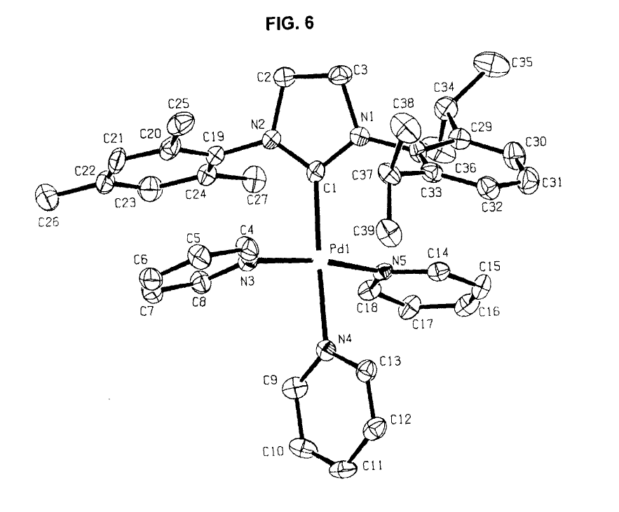

The X-ray crystal structure of compound IC is shown in FIG. 6.

### (iv) Compound ID

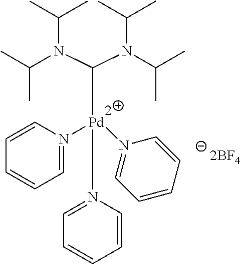

In the glovebox, a small vial was charged with the precursor A (50 mg, 0.106 mmol) and AgBF₄ (42 mg, 0.212 mmol) and CH₂Cl₂ was added to dissolve the mixture. 4 equiv. of pyridine (0.034 mL) was then added to the solution and the solution was allowed to stir overnight. The solvent was then concentrated to obtain a colorless solid. Yield: 69 mg, 91%. ¹H NMR (300 MHz, CD₂Cl₂): δ 7.30-9.10 (15H, m, pyridine), 3.65 (4H, m, (CH₃)₂CH₂), 1.38 (24H, m, CH₃). ¹⁹F NMR (282 MHz, CD₂Cl₂): δ −153 (s).

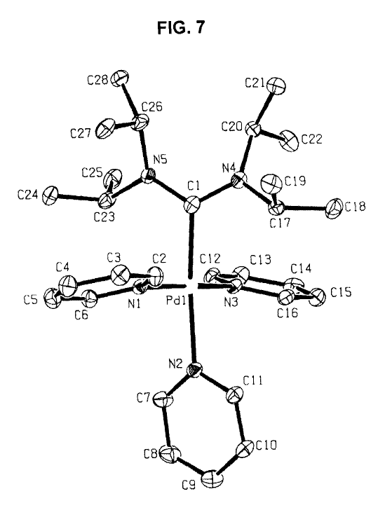

The X-ray crystal structure of compound ID is shown in FIG. 7.

### (v) Compound IE

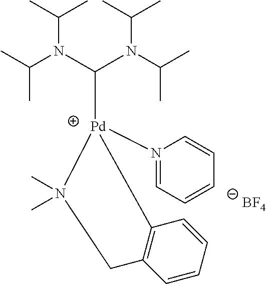

To a small vial was added the compound A (49 mg, 0.09 mmol) and pyridine (4 mL) was added to dissolve the compound. The solution was allowed to stir overnight. The solvent was then concentrated and the product recrystallized from ether to obtain a white solid, Yield: 44 mg, 78%. ¹H NMR (200 MHz, CD₂Cl₂): δ 8.70 (2H, d, pyridine), 8.00 (1H, m, pyridine), 7.62 (2H, m, pyridine), 7.00 (4H, m, Ph), 5.14 (4H, m, (CH₃)₂CH₂), 3.82 (2H, s, CH₃), 2.28 (6H, s, CH₃), 1.45 (12H, d, CH₃), 1.25 (12H, d, CH₃). ¹⁹F NMR (282 MHz, CD₂Cl₂): δ −153 (s).

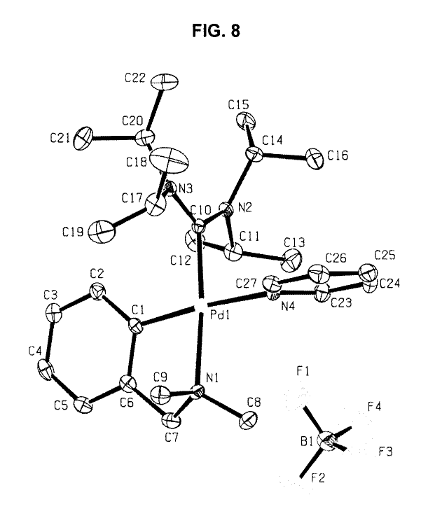

The x-ray crystal structure of compound IE is shown in FIG. 8.

### (vi) Compound IF

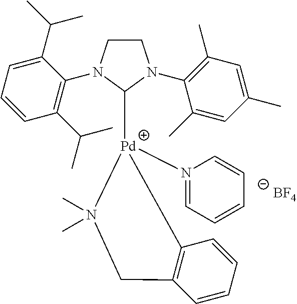

To a small vial was added the compound IB (86 mg, 0.13 mmol) and pyridine (4 mL) was added to dissolve the compound. The solution was allowed to stir overnight. The solvent was then concentrated and the product recrystallized from ether to obtain a white solid, Yield: 81 mg, 84%. ¹H NMR (200 MHz, CD₂Cl₂): δ 6.60-7.80 (14H, m, pyridine and Ph), 4.2 (6H, br m, CH₂), 3.45 (2H, m, CH₂), 2.25 (6H, s, CH₃), 1.84 (6H, d, CH₃), 1.50 (3H, s, CH₃), 1.20 (12H, m, CH₃). ¹⁹F NMR (282 MHz, CD₂Cl₂): δ −153 (s).

### (vi) Compound IG

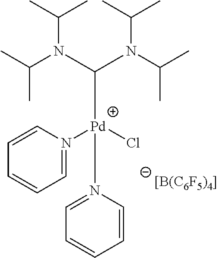

In the glovebox, a small vial was charged with the Precursor A (50 mg, 0.106 mmol) and [Li(OEt₂)_{2.5}][B(C₆F₅)₄] (100 mg, 0.117 mmol) and CH₂Cl₂ was added to dissolve the mixture. 1.1 equiv. of pyridine (˜0.01 mL) was then added to the solution and the solution was allowed to stir overnight. The precipitate was then filtered off and the filtrate was then concentrated and recrystallized from hexanes to obtain a pale yellow solid. Yield: 100 mg, 79%. ¹H NMR (300 MHz, CD₂Cl₂): δ 7.30-8.50 (10H, m, pyridine), 5.10 (4H, m, (CH₃)₂CH₂), 1.75 (12H, d, CH₃), 1.30 (12H, d, CH₃). ¹⁹F NMR (282 MHz, CD₂Cl₂): δ −133 (8F, s), −164 (8F, s), −168 (4F, s).

(vii) Compound IH

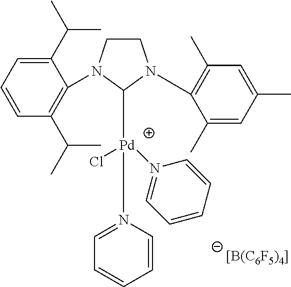

In the glovebox, a small vial was charged with the palladium compound (0.20 g, 0.33 mmol) and [Li(OEt₂)_{2.5}][B(C₆F₅)₄] (302 mg, 0.364 mmol) and CH₂Cl₂ was added to dissolve the mixture. 1.1 equiv. of pyridine (˜0.03 mL) was then added to the solution and the solution was allowed to stir overnight. The precipitate was then filtered off and the filtrate was then concentrated and recrystallized from hexanes to obtain a pale yellow solid. Yield: 440 mg, 99%. ¹H NMR (300 MHz, CD₂Cl₂): δ 6.85-8.50 (15H, m, pyridine and Ph), 4.0 (4H, m, CH₂), 2.6 (2H, s, (CH₃)₂CH₂), 1.80 (3H, s, CH₃), 1.60 (3H, s, CH₃), 1.25 (12H, m, CH₃), 0.8 (6H, m, CH₃). ¹⁹F NMR (282 MHz, CD₂Cl₂): δ −133 (8F, s), −164 (8F, s), −168 (4F, s).

(viii) Compound II

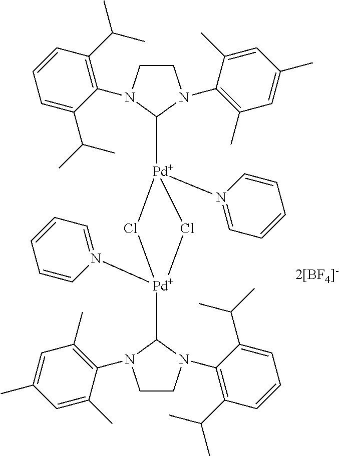

In the glovebox, dichloro(pyridine)[trans-(1,3-bis(2,6-diethylphenyl)imidazolin-2-ylidene)]palladium(II) (500 mg, 0.825 mmol) and AgBF₄ (160 mg, 0.825 mmol) were stirred in CH₂Cl₂ (20 mL) for 20 minutes. The resulting yellow solution was filtered through celite and evaporated to dryness. A beige-yellow solid was obtained. Yield: 400 mg, 74%. ¹H NMR (300 MHz, CD₂Cl₂): δ 7.90-6.80 (20H, m, pyridine and Ph), 4.20-3.80 (8H, m, CH₂), 2.40-2.20 (4H, s, CH), 1.60-0.80 (8H, m, CH₃). ¹⁹F NMR (282 MHz, CD₂Cl₂): δ −154 (s).

(vii) Compound IJ

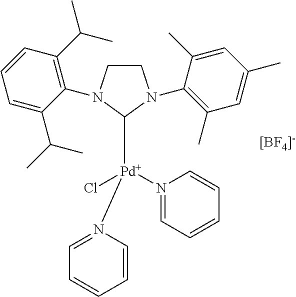

In air, compound II (100 mg, 0.150 mmol) and pyridine (4 mL) were stirred overnight. The resulting yellow solution was evaporated to dryness. The resulting bright yellow solid was recrystallized with CH₃CN/Hexanes. Yield: 90 mg, 90%. ¹H NMR (300 MHz, CD₂Cl₂): δ 7.90-6.80 (15H, m, pyridine and Ph), 4.20-3.80 (4H, m, CH₂), 2.60-1.80 (2H, m, CH), 1.60-0.80 (21H, m, CH₃), ¹⁹F NMR (282 MHz, CD₂Cl₂): δ −153 (s).

### Example 3

### Synthesis of Compound 3

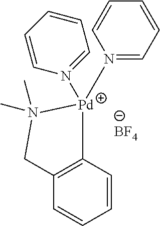

To a 100 mL flask was added Di-μ-chlorobis(N,N)-dimethylbenzylamine)dipalladium (0.97 g, 1.76 mmol) and 2 equiv. of AgBF₄ (0.699 g, 3.52 mmol) and CH₂Cl₂ was added to dissolve it. 4 equiv. of pyridine (0.567 ml, 7.04 mmol) was added. The precipitate formed was filtered off and the filtrate was concentrated to give a white color solid. Yield: 1.67 g, 82%. ¹H NMR (300 MHz, CD₂Cl₂): δ(2H, m, Py), 6.80-7.95 (11H, m, Ph and Py), 6.00 (1H, d, Ph), 4.12 (2H, s, CH₂), 2.62 (6H, s, CH₃). ¹⁹F NMR (282 MHz, CD₂Cl₂): δ −153 (s).

### Example 4

### Synthesis of Compound 4

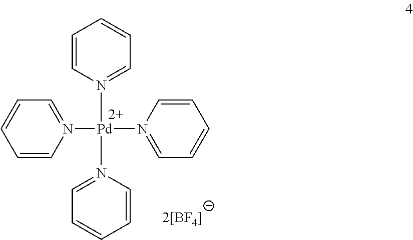

This compound was synthesized according to A the literature method.³ Yield: 80%.

### Example 5

### Synthesis of Compound 5

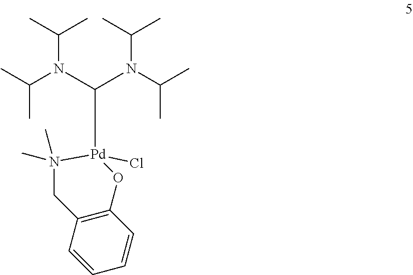

The oxygen-containing palladacycle precursor was synthesized according to the literature method.⁴ To a flask was added the palladacycle precursor (0.688 g, 1.177 mmol) and 2 equiv. of the free ADC-carbene (0.5 g, 2.35 mmol) and the solid mixture was dissolved in THF. The solution was allowed to stir overnight. The workup procedure was the same as B. Pale red solid. Yield: 0.15 g, 25%. ¹H NMR (200 MHz, CD₂Cl₂): d 7.05 (1H, m, Ph), 6.86 (1H, d, Ph), 6.68 (1H, d, Ph), 6.55 (1H, m, Ph), 5.22 (4H, m, (CH₃)₂CH₂), 3.18 (2H, s, CH₂), 2.59 (6H, s, CH₃), 1.40 (24H, d, CH₃).

### Example 6

### General Procedure for Suzuki-Miyaura Coupling

To a solution of an aryl halide (0.5 mmol), aryl boronic acid (0.60 mmol) and potassium (or cesium) carbonate (1 mmol, 2.0 equiv) in 1,4-dioxane or alcoholic solvent (2.0 mL) was added [Pd(Cl)(NHC)(py)]₂⁺BF₄⁻ (0.01 mmol, 2 mol %) under nitrogen or argon gas. The reaction was then stirred at 80° C. under reflux conditions for 16 hours, the mixture was then cooled to room temperature, filtered and concentrated in vacuo, the residue was subsequently purified by silica gel chromatography (hexanes/EtOAc or hexanes/ether). The isolated products were characterized by ¹H and ¹³C NMR spectroscopy.

### Discussion

Various structural motifs (i.e. bridging halogens, pyridine coordination to Pd—NHC complexes; and also the newly introduced ionic character) have been incorporated into the precatalysts reported herein. In Suzuki-Miyaura coupling the dimeric II, demonstrated good catalytic activity (as seen in Table 1). II was also used to couple a number of different substrate (as seen in Table 2). The use of Cs₂CO₃ as a base instead of K₂CO₃ was investigated (entry 14 and 15). The use of different alcoholic solvents was also investigated (Tables 3 and 4).

### Example 7

### General Procedure for Kumada Coupling

To a solution of aryl Grignard reagent (0.6 mmol) and organo halide (0.5 mmol) in THF was added 0.001 mmol of catalyst II under argon at room temperature. The reaction mixture was then stirred for 2 hours. After this time the reaction mixture was diluted with diethyl ether and filtered. The solvent was removed in vacuo and the residue was purified by silica gel chromatography.

### Example 8

### General Procedure for Negishi Coupling

To a solution of organozinc halide (0.6 mmol) and organo halide (0.5 mmol) in THF was added 0.001 mmol of catalyst II under argon at room temperature. The reaction mixture was then stirred for 2 hours. After this time the reaction mixture was diluted with diethyl ether and filtered. The solvent was removed in vacuo and the residue was purified by silica gel chromatography.

### Discussion

The results are shown in Tables 5 and 6. II was found to give good conversion at room temperature in THF. The addition of LiBr did not increase the sp²-sp² coupling (Table 5), but the conversions were doubled for the sp²-sp³ coupling (Table 6).

### Example 9

### General Procedure for Sonogashira Coupling

To a solution of organo halide (0.5 mmol), terminal alkyne (0.6 mmol) and a base (Cs₂CO₃/triethylamine), was added the catalyst II (0.01 mmol), in some cases CuI (0.01 mmol) was added as a co-catalyst and/or PPh₃ (0.5 mmol) was added as a co-ligand, and the reaction was purged with argon, heated to 80° C. and stirred at this temperature for 16 hours. The reaction mixture was allowed to cool to room temperature, diluted with diethyl ether, filtered, and the solvent removed in vacuo. The residue was purified by silica gel chromatography.

### Discussion

The results are shown in Table 7. It was found that the addition of PPh₃ as a co-ligand increases the yields while the addition of CuI in most cases leads to homocoupled product. DMF was the only solvent used with CuI that did not give homocoupled product. Changing the base from Cs₂CO₃ to triethylamine also led to the homocoupled product.

### Example 10

### General Procedure for Heck-Mizoroki Coupling

A solution of the alkene (0.50 mmol), aryl halide (0.5 mmol), [Pd(Cl)₂(NHC)(py)]₂ II (0.01 mmol) and potassium carbonate (1 mmol) in 1,4-dioxane (2.0 mL), in a pressure tube, was purged with argon, the pressure tube was then sealed with a screw cap; and the reaction was stirred for 16 hours at 100° C. The reaction mixture was then cooled to room temperature, filtered and concentrated in vacuo to afford the crude aryl-alkenyl derivative, which was subsequently purified by silica gel chromatography (hexanes/EtOAc or hexanes/ether). The results are shown in Table 8.

### Example 11

### Characterization Data

### (i) Biphenyl

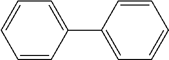 [SMILES: c1ccc(-c2ccccc2)cc1]

Isolated as colorless solid; ¹H NMR (CDCl₃, 300 MHz): δ 7.71 (4H, d, J=7.8 Hz), 7.55 (4H, t, J=7.5 Hz), 7.45 (2H, t, J=7.2 Hz).

### (ii) 4-methoxy-4′-methylbiphenyl

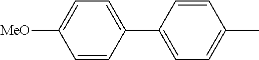

Isolated as yellow solid; ¹H NMR (CDCl₃, 300 MHz): δ 7.51 (2H, d, J=8.7 Hz), 7.45 (2H, d, J=7.8 Hz), 7.23 (2H, d, J=7.8 Hz), 6.97 (2H, d, J=8.7 Hz), 3.85 (3H, s), 2.39 (3H, s); ¹³C NMR (CDCl₃, 75 MHz): δ 159.19, 138.24, 136.62, 134.02, 129.72, 128.24, 126.87, 114.42, 55.63, 22.99.

### (iii) 4-methoxybiphenyl

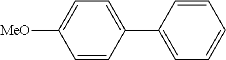 [SMILES: COc1ccc(-c2ccccc2)cc1]

Isolated as a colourless solid; ¹H NMR (CDCl₃, 300 MHz): δ 7.55-7.61 (4H, m), 7.42-7.48 (2H, tt), 7.34-7.36 (1H, tt), 6.99-7.03 (2H, dt), 3.87 (3H, s); ¹³C NMR (CDCl₃, 75 MHz): δ 159.94, 141.16, 134.12, 129.04, 128.47, 127.05, 126.97, 114.54, 55.65.

### (iv) 4′-methoxybiphenyl-4-carbaldehyde

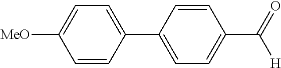 [SMILES: COc1ccc(-c2ccc(C=O)cc2)cc1]

Isolated as dark yellow solid; ¹H NMR (CDCl₃, 300 MHz): δ 10.04 (1H, s), 7.93, (2H, d, J=8.4 Hz), 7.72 (2H, d, J=8.4 Hz), 7.60, (2H, d, J=8.7 Hz), 7.01, (2H, d, J=8.7 Hz), 3.873 (3H, s); ¹³C NMR (CDCl₃, 75 MHz): δ 192.57, 131.02, 129.216, 127.77, 115.21, 56.13.

### (v) 1-(4′-methoxybiphenyl-4-yl)ethanone

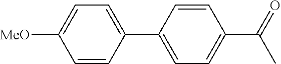 [SMILES: COc1ccc(-c2ccc(C(C)=O)cc2)cc1]

Isolated as yellow solid; ¹H NMR (CDCl₃, 300 MHz): δ 8.01, (2H, d, J=8.7 Hz), 7.63, (2H, d, J=8.7 Hz), 7.59, (2H, d, J=8.7 Hz), 7.00, (2H, d, J=8.7 Hz), 3.87 (3H, s), 2.63 (3H, s); ¹³C NMR (CDCl₃, 75 MHz): δ 198.18, 160.31, 145.78, 135.67, 132.65, 129.36, 128.76, 127.03, 114.81, 55.80, 31.13.

### (vi) 4-fluoro-4′-methoxybiphenyl

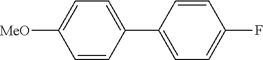 [SMILES: COc1ccc(-c2ccc(F)cc2)cc1]

Isolated as bright yellow solid; ¹H NMR (CDCl₃, 300 MHz): δ 7.46-7.52 (4H, m), 7.10 (2H, dd, J=8.7 Hz, 8.7 Hz), 6.98 (2H, dd, J=8.7 Hz, 8.7 Hz), 3.85 (3H, s); ¹³C NMR (CDCl₃, 75 MHz): δ 163.90, 159.28, 137.15, 133.03, 128.36 (d, J=7.5 Hz), 128.23, 115.63 (d, J=20.1 Hz), 114.43, 55.56.

### (vii) 2,4′-dimethoxybiphenyl

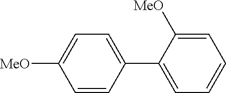 [SMILES: COc1ccc(-c2ccccc2OC)cc1]

Isolated as colourless solid; ¹H NMR (CDCl₃, 300 MHz): δ 7.47 (2H, d, J=8.7 Hz), 7.23-7.32 (2H, m), 6.93-7.03 (4H, m).

### (viii) 2,2′-dimethylbiphenyl

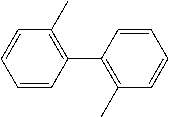

Isolated as yellow liquid; ¹H NMR (CDCl₃, 300 MHz): δ 7.28-7.21 (6H, m), 7.11 (2H, d, J=6.6 Hz), 2.06 (6H, s); ¹³C NMR (CDCl₃, 75 MHz): δ 141.68, 14.92, 129.90, 129.38, 127.25, 125.63, 29.83.

### (ix) 1,1′-(biphenyl-4,4′)diethanone

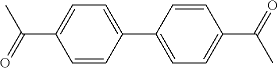 [SMILES: CC(=O)c1ccc(-c2ccc(C(C)=O)cc2)cc1]

Isolated as bright yellow; ¹H NMR (CDCl₃, 300 MHz): δ 7.811, (4H, d, J=7.2 Hz), 7.596, (4H, d, J=7.2 Hz), 2.597 (6H, s); ¹³C NMR (CDCl₃, 75 MHz): δ 197.15, 135.89, 131.99, 129.95, 128.42, 26.68.

### (x) (E)-methyl 3-(4-formylphenyl)acrylate

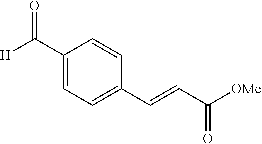 [SMILES: COC(=O)/C=C/c1ccc(C=O)cc1]

Isolated as dark yellow solid; ¹H NMR (CDCl₃, 300 MHz): δ 10.03 (1H, s), 7.90 (2H, d, J=8.4 Hz), 8.04-7.54 (3H, m), 6.56 (1H, d, J=15.9 Hz), 3.83 (3H, s); ¹³C NMR (CDCl₃, 75 MHz): δ 191.56, 166.94, 143.26, 140.17, 137.32, 132.02, 131.44, 130.31, 128.65, 121.11, 52.13.

### (xi) (E)-methyl-3-(4-acethylphenyl)acrylate

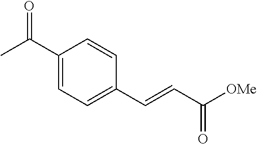

Isolated as a yellow-orange solid; ¹H NMR (CDCl₃, 300 MHz): δ 7.97 (2H, d, J=8.4 Hz), 7.71 (1H, d, J=15.9 Hz), 7.61 (2H, d, J=8.4 Hz), 6.53 (1H, d, J=15.9 Hz), 3.83 (3H, s), 2.62 (3H, s); ¹³C NMR (CDCl₃, 75 MHz): δ 143.45, 138.83, 138.16, 129.00, 128.28, 120.46, 52.05, 26.83.

### (xii) (E)-methyl 3-(4-methoxyphenyl)acrylate

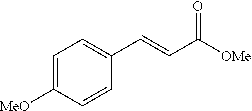 [SMILES: COC(=O)/C=C/c1ccc(OC)cc1]

Isolated as pale yellow crystalline solid; ¹H NMR (CDCl₃, 300 MHz): δ 7.65, (1H, d, J=15.9 Hz), 7.47, (2H, d, J=9 Hz), 7.91, (2H, d, J=9 Hz), 6.31, (1H, d, J=15.9 Hz), 3.84 (3H, s), 3.79 (3H, s); ¹³C NMR (CDCl₃, 75 MHz): δ 167.93, 161.52, 144.68, 129.88, 127.25, 115.39, 114.45, 55.51, 51.73.

### (xiii) (E)-tert-butyl 3-(4-formylphenyl)acrylate

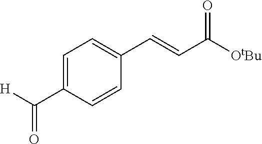 [SMILES: CC(C)(C)OC(=O)/C=C/c1ccc(C=O)cc1]

Isolated as a pale white solid; ¹H NMR (CDCl₃, 300 MHz): δ 10.01, (1H, s), 7.87, (2H, d, J=8.4 Hz), 7.64, (2H, d, J=8.4 Hz), 7.59 (1H, d, J=15.9 Hz), 6.47 (1H, d, J=15.9 Hz), 1.53 (9H, s); ¹³C NMR (CDCl₃, 75 MHz): δ 191.67, 165.86, 142.03, 140.70, 137.27, 130.38, 128.38, 123.73, 81.30, 28.42.

### (xiv) (E)-tert-butyl 3-(4-methoxyphenyl)acrylate

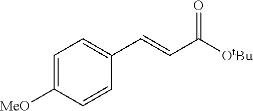 [SMILES: COc1ccc(/C=C/C(=O)OC(C)(C)C)cc1]

Isolated as clear crystals; ¹H NMR (CDCl₃, 500 MHz): δ 7.54, (1H, d, J=16 Hz), 7.46, (2H, d, J=8.5 Hz), 6.89 (2H, d, J=8.5 Hz), 6.24 (1H, d, J=16 Hz), 3.83 (3H, s), 1.53 (9H, s); ¹³C NMR (CDCl₃, 75 MHz): δ 166.97, 161.37, 143.47, 129.81, 127.66, 117.97, 114.51, 80.50, 55.62, 28.51.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

TABLE 1
Suzuki-Miyaura Coupling

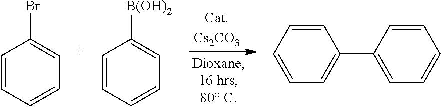

| Entry | Catalyst | % conv. |
|---|---|---|
| 1 | A | 79 |
| 2 | B | 81 |
| 3 | C | 12 |
| 4 | IA | 44 |
| 5 | IB | 15 |
| 6 | IC | 29 |
| 7 | ID | 73 |
| 8 | IE | 64 |
| 9 | IF | 35 |
| 10 | II | 83 |
| 11 | 1J | 86 |
| 12 | No Cat., with base | 0 |
| 13 | No Cat., No base | 27 |
| 14 | II, No base | 27 |

TABLE 2
Suzuki-Miyaura Coupling (Using Catalyst II)

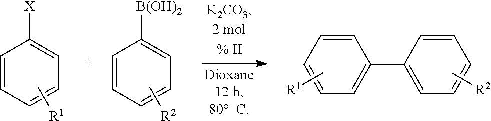

| Entry | X | R1 | R2 | % conv. |
|---|---|---|---|---|
| 1 | Cl | 4-OMe | 4-Me | 34 |
| 2 | Br | 4-OMe | 4-Me | 48 |
| 3 | Br | 2-Me | 1-napthalene | 19 |
| 4 | Cl | — | 4-OMe | 76 |
| 5 | Br | — | 3-OMe | 23 |
| 6 | Br | 4-CHO | 4-OMe | 24 |
| 7 | Br | 4-C(O)Me | 4-OMe | 9 |
| 8 | Br | 2-Me | 4-F | 5 |
| 9 | Br | 4-OMe | 4-OMe | 17 |
| 10 | Br | 4-OMe | 2-OMe | 14 |
| 11 | Br | 2-Me | 2-Me | 69 |
| 12 | Br | 4-C(O)Me | 4-C(O)Me | 48 |
| 13 | Br | 4-OMe | 2-CF₃ | 44 |
| 14^{a} | Br | 4-OMe | 4-Me | 86 |
| 15^{a} | Cl | — | 4-OMe | 88 |
| 16 | Br | — | — | 83 |
| 17^{b} | Cl | — | — | 87 |

^{a}:Cs₂CO₃ was used as a base instead of K₂CO₃
^{b}:ⁱPrOH was used as solvent

TABLE 3
Suzuki-Miyaura Coupling (Using EtOH and ⁱPrOH as solvent)

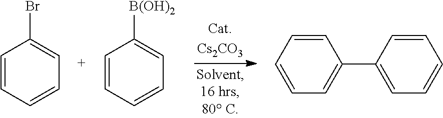

| Entry | Catalyst | EtOH | ⁱPrOH |
|---|---|---|---|
| 1 | A | 87 | 77 |
| 2 | B | 83 | 85 |
| 3 | C | 87 | 87 |
| 4 | IA | 90 | 82 |
| 5 | IB | 84 | 88 |
| 6 | IC | 88 | 87 |
| 7 | ID | 55 | 70 |
| 8 | IE | 86 | 82 |
| 9 | IF | 80 | 79 |
| 10 | II | 88 | 88 |
| 11 | II | 47 | 36 |
| | No Base | | |
| 12 | Base, No Cat. | 35 | 11 |

TABLE 4
Suzuki-Miyaura Coupling (Comparision between EtOH and
ⁱPrOH towards PhCl substrate)

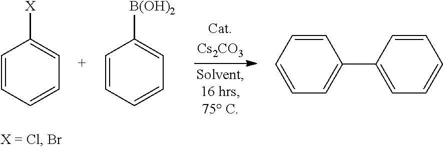

| | | EtOH | ⁱPrOH | EtOH | ⁱPrOH |
|---|---|---|---|---|---|
| Entry | Catalyst | PhBr | PhBr | PhCl | PhCl |
| 1 | A | 79 | 78 | 47 | 82 |
| 2 | B | 90 | 84 | 40 | 65 |
| 3 | C | 84 | 90 | 76 | 85 |
| 4 | D | 52 | 75 | 29 | 60 |
| 5 | IA | 81 | 88 | 14 | 63 |
| 6 | II | 90 | 90 | 73 | 87 |

TABLE 5
Sp²-Sp² Negishi Coupling Catalyzed by Compound II

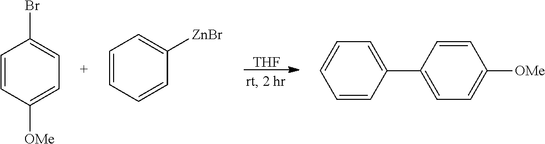

| Entry | Additives | Conversion (%) |
|---|---|---|
| 1 | — | 80 |
| 2 | LiBr | 83 |

TABLE 6
Sp²-Sp³ Negishi Coupling Catalyzed by Compound II

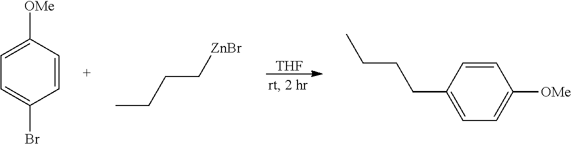

| Entry | Additives | Conversion (%) |
|---|---|---|
| 1 | — | 34 |
| 2 | LiBr | 62 |

TABLE 7
Sonogashira Coupling

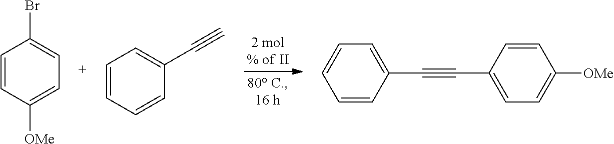

| Entry | Solvent | CuI | PPh₃ | Conversion (%) |
|---|---|---|---|---|
| 1 | DMF | Added | — | 17 |
| 2 | DMF | Added | Added | 41 |
| 3 | Dioxane | — | — | 26 |
| 4 | DMF | — | Added | 32 |
| 5 | Dioxane | — | Added | 47 |
| 6 | DME | — | Added | 48 |

TABLE 8
Heck-Mizoroki Coupling

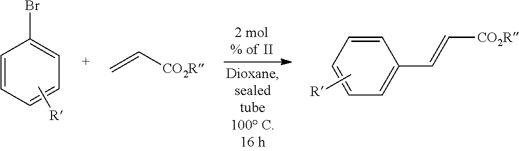

| Entry | R′ | R″ | Conversion (%) |
|---|---|---|---|
| 1 | 4-OMe | ^{t}Bu | 50 |
| 2 | 4-CHO | ^{t}Bu | quant |
| 3* | 4-CHO | Me | 23 |
| 4* | 4-C(O)Me | Me | 22 |
| 5* | 2-Me | Me | 11 |

*: reactions run under reflux conditions

1. K. C. Nicolaou, P. G. Bulger, D. Sarlah, Angew. Chem. Int. Ed., 2005, 44, 4442-4489.
2. A. Yokoyama, H. Suzuki, Y. Kubota, K. Ohuchi, H. Higashimura and T. Yokozawa, J. Am. Chem. Soc., 2007, 129, 7236-7237; S. L. Hargreaves, B. L. Pilkington, S. E. Russell and P. A. Worthington, Tetrahedron Lett., 2000, 41, 1653-1656.
3. A. De Léon, J. Pons, X. Solans and M. Font-Bardia, Acta Cryst., 2007, E63, m2164.
4. P. L. Alster, H. T. Teunissen, J. Boersma, A. L. Spek and G. van Koten, Organomet. 1993, 12, 4691.

1. A compound of the formula (I)
[Pd(X)_{q}(LB)ₙ(LBX)ₜ(DC)]^{r+}[Y^{m-}]ₚ (I)
wherein
DC is a diamino carbene ligand,
X is any anionic ligand,
LB is any neutral Lewis base,
LBX is a combined anionic and neutral ligand,
Y is any non-coordinating anion,
q is 0 or 1,
n is 0 to 3,
t is 0 or 1,
r is 1 or 2,
m is 1 or 2,
p is 1 or 2,
wherein the sum of q+r is 2 or t+r is 2,
when t is 1, q is 0,
when r is 1, m and p are both 1, and
when r is 2, either (i) m is 2 and p is 1, or (ii) m is 1 and p is 2,
wherein when p is 2, Y is the same or different, and
wherein the compound of the formula (I) is chiral or achiral.

2. A compound of the formula (Ia):
[Pd(X)_{q}(LB)ₙ(LBX)ₜ(DC)]₂^{a+}[V⁻]ᵤ[Z²⁻]_{y} (Ia)
wherein
DC is a diamino carbene ligand,
X is any anionic ligand,
LB is any neutral Lewis base,
LBX is a combined anionic and neutral ligand,
V is any non-coordinating mono-anion,
Z is any non-coordinating di-anion
q is 0 or 1,
n is 0 to 3,
t is 0 or 1,
a is 2 or 4,
u is 0, 2 or 4,
y is 0, 1 or 2,
wherein the sum of q+a is 3 or 4, or t+a is 3 or 4,
when t is 1, q is 0,
when a is 2, either (i) u is 2 and y is 0; or (ii) u is 0 and y is 1; or
when a is 4, either (i) u is 4 and y is 0; (ii) u is 2 and y is 1; or (iii) u is 0 and y is 2;
wherein when u is 2 or 4, V is the same or different, and
when y is 2, Z is the same or different, and
wherein the compound of the formula (Ia) is chiral or achiral.

3. The compound according to claim 1, wherein the diamino carbene ligand is of the formula (II): 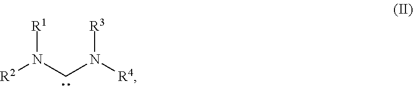
wherein
R¹, R², R³ and R⁴ are independently selected from H, C₁₋₂₀alkyl, C₂₋₂₀alkenyl, C₂₋₂₀alkynyl, C₃₋₂₀cycloalkyl, heteroaryl and aryl, each group being optionally substituted, or
R¹ and R² and/or R³ and R⁴ are linked to form, together with the nitrogen atom to which they are attached, an optionally substituted monocyclic or polycyclic, saturated or unsaturated ring system that contains 3 to 20 carbon atoms, of which one or more of the carbon atoms is optionally replaced with a heteromoiety selected from O, S, NH and NC₁₋₆alkyl, and/or
R¹ and R³ or R² and R⁴ are linked to form, together with the nitrogen atoms to which they are attached, an optionally substituted monocyclic or polycyclic, saturated or unsaturated ring system that contains 3 to 20 carbon atoms, of which one or more of the carbon atoms is optionally replaced with a heteromoiety selected from O, S, NH and NC₁₋₆alkyl, the optional substituents on R¹, R², R³ and R⁴ are independently selected from one or more of C₁₋₆alkyl, halo, halo-substituted C₁₋₆alkyl, C₃₋₁₀cycloalkyl, aryl and heteroaryl, and wherein the compound of the formula (II) is chiral or achiral.

4. The compound according to claim 3, wherein R¹, R², R³ and R⁴ are independently selected from H, C₁₋₁₀alkyl, C₂₋₁₀alkenyl, C₂₋₁₀alkynyl, C₃₋₁₀cycloalkyl, heteroaryl and aryl, each group being optionally substituted.

5. (canceled)

6. The compound according to claim 3, wherein R¹, R², R³ and R⁴ are independently selected from H, C₂₋₄alkenyl, C₂₋₆alkynyl, C₅₋₆cycloalkyl and phenyl, each group being optionally substituted.

7. (canceled)

8. The compound according to claim 3, wherein R¹, R², R³ and R⁴ are independently selected from H, methyl, ethyl, propyl, isopropyl, butyl and phenyl, each group being optionally substituted.

9. (canceled)

10. The compound according to claim 3, wherein R¹, R², R³ and R⁴ are independently selected from

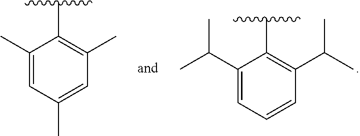

11. The compound according to claim 3, wherein R¹ and R² and/or R³ and R⁴ are linked to form, together with the nitrogen atom to which they are attached, an optionally substituted monocyclic or polycyclic, saturated or unsaturated ring system that contains 3 to 10 carbon atoms, of which one or more of the carbon atoms is optionally replaced with a heteromoiety selected from O, S, NH and NC₁₋₆alkyl.

12. (canceled)

13. The compound according to claim 11, wherein R¹ and R² and/or R³ and R⁴ are linked to form, together with the nitrogen atom to which they are attached, an optionally substituted monocyclic, saturated or unsaturated ring system that contains 5 to 6 carbon atoms, of which one or more of the carbon atoms is optionally replaced with a heteromoiety selected from O, S, NH and NC₁₋₆alkyl.

14. The compound according to claim 3, wherein R¹ and R³ or R² and R⁴ are linked to form, together with the nitrogen atoms to which they are attached, an optionally substituted monocyclic or polycyclic, saturated or unsaturated ring system that contains 3 to 10 carbon atoms, of which one or more of the carbon atoms is optionally replaced with a heteromoiety selected from O, S, NH and NC₁₋₆alkyl.

15. (canceled)

16. The compound according to claim 14, wherein R¹ and R³ or R² and R⁴ are linked to form, together with the nitrogen atoms to which they are attached, an optionally substituted monocyclic, saturated or unsaturated ring system that contains 5 to 6 carbon atoms, of which one or more of the carbon atoms is optionally replaced with a heteromoiety selected from O, S, NH and NC₁₋₆alkyl.

17. The compound according to claim 3, wherein the compound of the formula (II) is

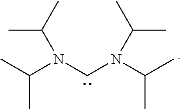

18. The compound according to claim 3, wherein the compound of the formula (II) is

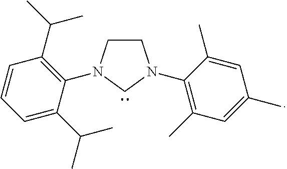

19. (canceled)

20. The compound according to claim 1, wherein the anionic ligand X is halo, H, C₁₋₆alkoxy or carboxyl.

21. (canceled)

22. The compound according to claim 1, wherein LB is acetonitrile or pyridine.

23. The compound according to claim 1, wherein LBX is

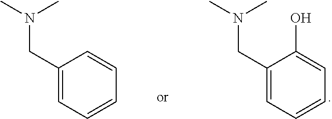

24. The compound according to claim 1, wherein Y and V are BF₄, B(C₆F₅)₄ or a carborane and Z is CO₃, SO₄ or C₂O₄.

25. The compound according to claim 1, wherein the compound of the formula (I) or (Ia) is

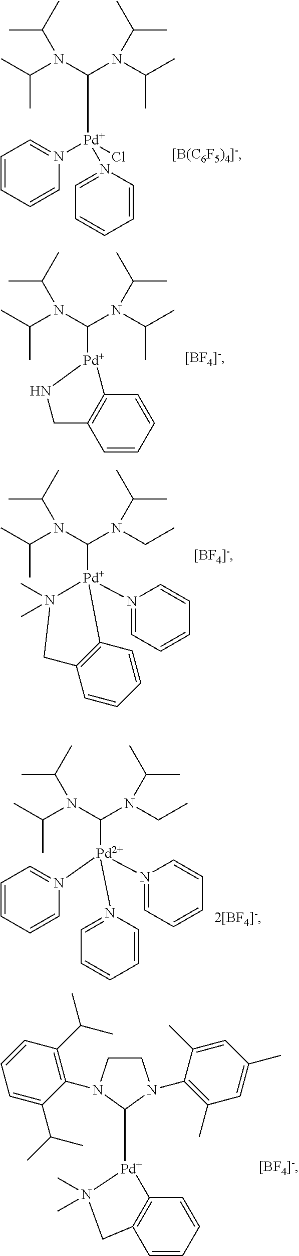

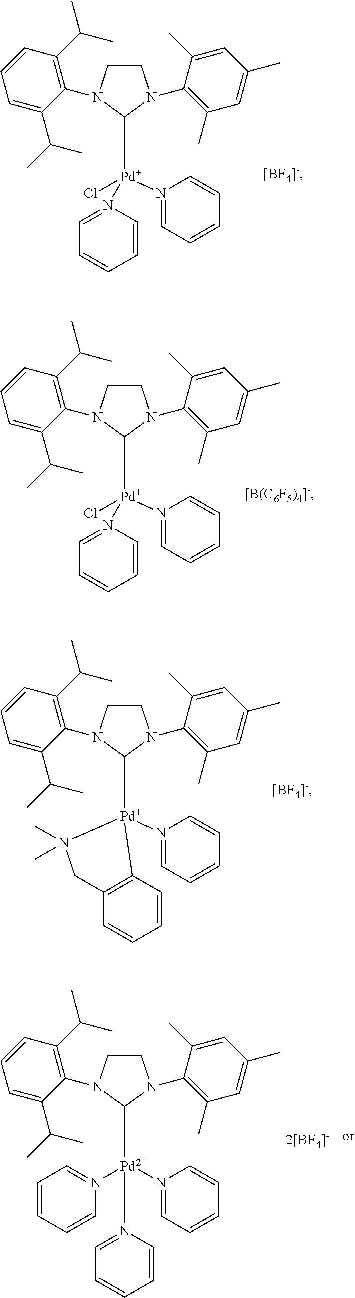

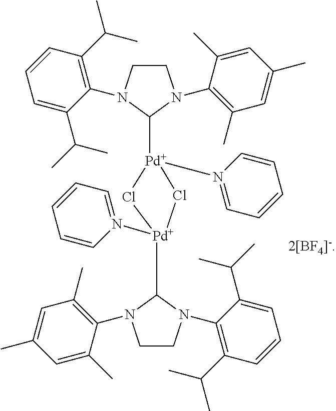

26. (canceled)

27. A method of performing palladium-catalyzed organic synthesis reactions comprising contacting substrates for the organic synthesis reaction with a compound of the formula (I) as defined in claim 1 in the presence of a base under conditions for performing the organic synthesis reaction, and optionally isolating one or more products from the organic synthesis reaction.

28. The method according to claim 27, wherein the organic synthesis reaction is cross-coupling reaction or hydroamination reaction.

29. The method according to claim 28, wherein the reaction is a Suzuki-Miyaura coupling, Kumada coupling, Negishi coupling, Sonogashira coupling, Hartwig-Buchwald amination or a Heck-Mizoroki coupling.
